(19) United States
(12) Patent Application Publication
(10) Pub. No.: US 2024/0409977 A1
(43) Pub. Date: Dec. 12, 2024

(54) METHOD FOR PREPARING BETA-CAROTENE FROM PEPPER PIGMENT AND PRODUCT THEREOF

(71) Applicant: HENAN ZHONGDA HENGYUAN BIOTECHNOLOGY STOCK CO., LTD., Luohe City (CN)

(72) Inventors: Yihong LIU, Luohe City (CN); Ziheng JIN, Luohe City (CN); Linzheng LI, Luohe City (CN); Yanjun WEN, Luohe City (CN); Tianyi PAN, Luohe City (CN); Xuejiao CHEN, Luohe City (CN); Xiaoka HU, Luohe City (CN); Yang LIU, Luohe City (CN)

(21) Appl. No.: 18/377,754

(22) Filed: Oct. 6, 2023

(30) Foreign Application Priority Data
Jun. 9, 2023 (CN) 202310680024.5

(51) Int. Cl. C12P 23/00; C12N 9/20; C12P 5/00

(52) U.S. Cl. CPC C12P 23/00; C12N 9/20; C12P 5/007; C12Y 301/01

(57) ABSTRACT

A method for preparing beta-carotene from pepper pigment includes: (1) enzymatic hydrolysis of paprika oleoresin to obtain a paprika red pigment hydrolysate; (2) extraction of the paprika red pigment hydrolysate to obtain a paprika red pigment solution; (3) refined filtration to obtain a paprika red pigment filtrate; (4) adsorption: carrying out chromatographic separation with a medium-pressure silica gel chromatographic column, and collecting an effluent from the chromatographic column; (5) elution of beta-carotene; (6) elution of yellow pigments; (7) elution of red pigments; and (8) elution of orange pigments. The invention combines enzymatic hydrolysis with medium-pressure column chromatography for the first time, where chromatographic silica gel with smaller particle size is used for adsorption, and four-stage elution treatment is carried out, so that beta-carotene with a content of more than 20% can be directly separated, and at the same time, reddish paprika red pigment with higher quality is prepared.

### FIELD OF THE INVENTION

The invention relates to the technical field of food additive refining, and specifically to a method for preparing beta-carotene from pepper pigment and a product thereof.

### DESCRIPTION OF THE RELATED ART

Beta-carotene (C₄₀H₅₆) is one of the carotenoids and an orange fat-soluble compound. It is the most ubiquitous natural pigment in nature. For example, green vegetables, sweet potatoes, carrots, peppers, Dunaliella salina, palms, and the like are rich in beta-carotene.

There are three main sources of beta-carotene: synthesis, microbial fermentation, and extraction from natural plants. Because beta-carotene is good for people's health, but the amount of beta-carotene from natural sources cannot meet the market demand, chemical synthesis of beta-carotene was very popular. However, carotene synthesized by chemical methods cannot be completely absorbed by human body, has toxic and side effects on human body, and will cause irreversible pathological changes to human body after long-term use, so it is not accepted by most food scientists, and has been banned as food additives in western developed countries.

Naturally extracted carotene is easy to be absorbed by human body and has good antioxidant capacity, and therefore is favored by customers. At present, palm oil and algae such as Dunaliella salina are good raw materials of carotene. The key point of obtaining natural carotene from Dunaliella salina is the cultivation of Dunaliella salina. However, the cultivation of Dunaliella salina has strict requirements on external environmental conditions, making it difficult to expand the scale of cultivation. Therefore, the output of natural carotene from Dunaliella salina cannot meet the international market demand. The development of natural beta-carotene in China is seriously limited because the main producing area of palm oil is in Southeast Asia.

At present, pepper is the raw material that can be used as the source of beta-carotene and has abundant yield in China. The annual output of pepper for pigment extraction in Xinjiang Province alone is over 300,000 tons. Paprika red pigment contained in pepper is mainly used in food, beverage, cosmetics, medicine and other industries, and is recognized as the best Class A red pigment in the world, with its color intensity being 10 times those of other pigments. The main components of pepper are capsanthin, capsorubin, beta-carotene, and zeaxanthin. Beta-carotene account for 13.9% or more of the total pigment content. However, because pepper is mostly used in food and other fields, its added value is too low, affecting the long-term development of the whole pepper industry.

In the prior art, due to the complex pigment composition of paprika red pigment and the difficulty of separation, separation technologies for paprika red pigment cannot be industrialized. Especially for the separation of beta-carotene from paprika red pigment, existing schemes of separating beta-carotene by preparative high performance liquid chromatography are applied only to the preparation of reference substances, require expensive separation materials and a complex elution system, and therefore are not suitable for industrial production. Moreover, these schemes also involve complex saponification treatment before separation, which leads to the transformation of the main components of paprika red pigment, e.g., the hydrolysis of capsanthin, ester derivatives of capsanthin, lutein ester and other substances, failing to ensure the natural characteristics of the remaining pigments after separation.

In addition, Chinese Patent Application No. CN106631953 discloses a method of preparing beta-carotene from pepper, where pepper pigment is separated into yellowish and reddish colors by normal-pressure silica gel chromatography, and then the yellowish paprika red pigment is treated by saponification and other refining methods to finally obtain beta-carotene. However, the column chromatography used in the process is not suitable for continuous production. For each batch, it is necessary to unpack the column, activate silica gel at high temperature, and then repack the column for separation. Moreover, the elution period at low pressure is long, the separation efficiency is low, the process is complex, and the production cycle is long, affecting the efficiency of separating beta-carotene from paprika red pigment.

To sum up, how to separate beta-carotene with high yield and purity from paprika red pigment is an urgent problem to be solved by those skilled in the art.

### SUMMARY OF THE INVENTION

An objective of the invention is to provide a method for preparing beta-carotene from pepper pigment, which uses a combination of enzymatic hydrolysis and medium-pressure column chromatography to prepare beta-carotene from paprika red pigment. The method combines enzymatic hydrolysis with medium-pressure column chromatography for the first time, where chromatographic silica gel with smaller particle size is used for adsorption, and four-stage elution treatment is carried out, so that beta-carotene with a content of 20% or more can be directly separated from paprika oleoresin having been subjected to enzymatic hydrolysis, with a yield of up to 92.5%, and at the same time, reddish paprika red pigment with higher quality is prepared.

To achieve the above objective, the invention provides a method for preparing beta-carotene from pepper pigment, which specifically includes the following steps:
(1) enzymatic hydrolysis of paprika oleoresin to obtain a paprika red pigment hydrolysate;
(2) extraction: dissolving the paprika red pigment hydrolysate obtained in the step (1) in an alkane solvent, standing for layering, and collecting the supernatant to obtain a paprika red pigment solution;
(3) refined filtration: filtering the paprika red pigment solution obtained in the step (2) by a filter membrane to obtain a paprika red pigment filtrate;
(4) adsorption: carrying out chromatographic separation of the paprika red pigment filtrate obtained in the step (3) with a medium-pressure silica gel chromatographic column, and collecting an effluent from the chromatographic column;
(5) elution of beta-carotene: after loading onto the medium-pressure chromatographic column is completed, eluting with a first eluent, and collecting and concentrating the eluate to obtain beta-carotene oleoresin;
(6) elution of yellow pigments: after the elution of beta-carotene is completed, eluting with a second eluent, and concentrating the eluate to obtain a yellow pigment oleoresin;
(7) elution of red pigments: after the elution of yellow pigments is completed, eluting with a third eluent, and concentrating the eluate to obtain reddish paprika red pigment; and
(8) elution of orange pigments: after the elution of red pigments is completed, eluting the silica gel column with a fourth eluent, concentrating the eluate, and mixing the concentrated eluate with the yellow pigment oleoresin to obtain orange paprika red pigment.

In a preferred implementation, after the step (8) is completed, the method further includes a step (9) of solvent replacement and equilibrium:
treating the medium-pressure silica gel chromatographic column by solvent replacement and equilibrium using a replacement solvent, to allow for separation production of a next batch.

In a preferred implementation, in the step 1, the enzymatic hydrolysis specifically includes: adding membrane filtration water and lipase into the paprika oleoresin, stirring and reacting the mixture.

In a preferred implementation, in the step (1), the mass ratio of the paprika oleoresin, the membrane filtration water, and the lipase is 1:(0.4-0.6):(0.01-0.02).

More preferably, the mass ratio of the paprika oleoresin, the membrane filtration water, and the lipase is 1:0.5:0.15.

Most preferably, a conductivity of the membrane filtration water is less than 20 μs/cm.

In a preferred implementation, in the step (1), the stirring and reaction are carried out at 35° C. to 40° C. for 6 h to 10 h. More preferably, the stirring and reaction are carried out at 38° C. for 10 h.

In the invention, after the paprika oleoresin is enzymatically hydrolyzed by a suitable lipase, oils and fats in the paprika oleoresin are converted into fatty acids by enzymatic hydrolysis to change their polarity, thereby enhancing the effect of separating beta-carotene from impurities including oils and fats.

In a preferred implementation, in the step (2), the alkane solvent is an alkane containing 4-7 carbon atoms.

More preferably, the alkane solvent containing 4-7 carbon atoms includes one or more of n-hexane, petroleum ether, and a vegetable oil extraction solvent.

In a preferred implementation, in the step (2), the alkane solvent is added in an amount to adjust the color value of the paprika red pigment solution to 60-120.

More preferably, the alkane solvent is added in an amount to adjust the color value of the paprika red pigment solution to 80-100.

In the invention, the alkane solvent is used as the extraction solvent, so that paprika red pigment can be directly extracted from the water phase system, thereby achieving an oil-water separation effect.

In a preferred implementation, in the step (3), the function of the refined filtration is to physically remove common impurities from the solution, and therefore may be carried out using a conventional method known to those skilled in the art. Preferably, the filter membrane for refined filtration is an organic filter membrane of 0.45 to 10 μm. More preferably, the filter membrane for refined filtration is an organic filter membrane of 1 μm.

In a preferred implementation, in the step (4), the stationary phase of the medium-pressure silica gel chromatographic column is 300-500 mesh chromatographic silica gel, and the mass ratio of the amount of silica gel to the paprika oleoresin is 1:(0.25-0.4).

In a preferred implementation, in the step (4), the pressure of the medium-pressure silica gel chromatographic column is 0.5-5 MPa, and the pigment adsorption amount is 40-120 color value/g.

More preferably, the pressure of the medium-pressure silica gel chromatographic column is 0.5-2 MPa, and the pigment adsorption amount is 60-100 color value/g.

In this step, of the components of the paprika red pigment are adsorbed by silica gel, and the process is accompanied by elution of beta-carotene and other substances. Because beta-carotene has the lowest polarity in the paprika red pigment, it is the first component to be eluted off.

In a preferred implementation, in the step (5), the first eluent is an alkane containing 4-7 carbon atoms, the elution volume is 1-5 BV, and the elution flow rate is 0.5-3 BV/h.

More preferably, the first eluent includes one or more of n-hexane, petroleum ether, and a vegetable oil extraction solvent, the elution volume is 1-3 BV, and the elution flow rate is 0.5-2 BV/h.

In a preferred implementation, in the step 5, the concentration is carried out at a temperature of 40° C. to 100° C.; and the concentration is carried out at a vacuum degree of −0.03 MPa to −0.09 MPa.

More preferably, the concentration is carried out at a temperature of 40° C. to 80° C.; and the concentration is carried out at a vacuum degree of −0.05 MPa to −0.09 MPa.

In this step, the alkane eluent with low polarity is used to realize elution and separation of beta-carotene, thereby improving the product yield.

In a preferred implementation, in the step (6), the second eluent is obtained by mixing an alkane containing 4-7 carbon atoms and acetone according to a mass ratio of (8-50):1.

More preferably, the alkane containing 4-7 carbon atoms includes one or more of n-hexane, petroleum ether, and a vegetable oil extraction solvent.

Most preferably, the mass ratio of the alkane containing 4-7 carbon atoms to acetone is (8-20):1.

In a preferred implementation, in the step (6), the elution volume is 1-5 BV, and the elution flow rate is 0.5-3 BV/h.

More preferably, the elution volume is 1-3 BV, and the elution flow rate is 0.5-2 BV/h.

In a preferred implementation, in the step 6, the concentration is carried out at a temperature of 40° C. to 100° C.; and the concentration is carried out at a vacuum degree of −0.03 MPa to −0.09 MPa.

More preferably, the concentration is carried out at a temperature of 40° C. to 80° C.; and the concentration is carried out at a vacuum degree of −0.05 MPa to −0.09 MPa.

During the elution of beta-carotene, yellow pigment also moves. However, the elution efficiency of the first eluent is low and the elution time is too long, so it is necessary to increase the polarity of the eluent to the second eluent for elution to realize the elution and separation of yellow pigment. Therefore, the elution solvent and elution conditions are designed.

In a preferred implementation, in the step (7), the third eluent is obtained by mixing an alkane containing 4-7 carbon atoms and acetone according to a mass ratio of (8-50):1.

More preferably, the alkane containing 4-7 carbon atoms includes one or more of n-hexane, petroleum ether, and a vegetable oil extraction solvent.

Most preferably, the mass ratio of the alkane containing 4-7 carbon atoms to acetone is (8-20):1.

In a preferred implementation, in the step (7), the elution volume is 1-5 BV, and the elution flow rate is 0.5-3 BV/h.

More preferably, the elution volume is 1-3 BV, and the elution flow rate is 0.5-2 BV/h.

In a preferred implementation, in the step 7, the concentration is carried out at a temperature of 40° C. to 100° C.; and the concentration is carried out at a vacuum degree of −0.03 MPa to −0.09 MPa.

More preferably, the concentration is carried out at a temperature of 40° C. to 80° C.; and the concentration is carried out at a vacuum degree of −0.05 MPa to −0.09 MPa.

In the step of elution of red pigments, to separate the reddish pigment from the orange pigment, it is not appropriate to further increase the polarity of the eluent, and the polarity of the second eluent satisfies the elution of the reddish pigment. In addition, the third eluent and the second eluent produce a difference in elution effect by adjusting the elution time (i.e., the eluent dosage).

In a preferred implementation, in the step (8), the fourth eluent is acetone, the elution volume is 1-5 BV, and the elution flow rate is 0.5-3 BV/h.

More preferably, the elution volume is 1-3 BV, and the elution flow rate is 0.5-2 BV/h.

In a preferred implementation, in the step 8, the concentration is carried out at a temperature of 40° C. to 100° C.; and the concentration is carried out at a vacuum degree of −0.03 MPa to −0.09 MPa.

More preferably, the concentration is carried out at a temperature of 40° C. to 80° C.; and the concentration is carried out at a vacuum degree of −0.05 MPa to −0.09 MPa.

In this step, the residual orange pigment and non-color impurities are directly eluted off by acetone with high polarity, to obtain high-quality reddish paprika red pigment.

In a preferred implementation, in the step (9), the replacement solvent is an alkane containing 4-7 carbon atoms, the solvent replacement and equilibrium volume is 1.5-5 BV, and the flow rate is 0.5-3 BV/h.

More preferably, the replacement solvent is an alkane containing 4-7 carbon atoms, the solvent replacement and equilibrium volume is 2-4 BV, and the flow rate is 0.5-2 BV/h.

Another objective of the invention is to provide beta-carotene prepared by any one of the above methods. In the invention, beta-carotene is directly separated from paprika oleoresin having been subjected to enzymatic hydrolysis by medium-pressure column chromatography, and at the same time, reddish paprika red pigment with higher quality is prepared. Continuous medium-pressure silica gel column chromatography is applied to the industrial separation and production of paprika red pigment for the first time, to achieve the industrialization of the direct separation of beta-carotene with a content of 20% or more from paprika red pigment. The overall process is simple and efficient. The yield of the beta-carotene obtained is 92.5% or above, with a purity of 20% or above.

Compared with the prior art, the technical solutions of the invention have the following advantages.

The invention combines, for the first time, the enzymatic hydrolysis technology with the medium-pressure column chromatographic separation and purification technology using chromatographic silica gel as the stationary phase, to directly separate beta-carotene from paprika oleoresin. To be specific, the paprika oleoresin is enzymatically hydrolyzed to obtain a paprika red pigment hydrolysate. The paprika red pigment hydrolysate is dissolved in a solvent, and subjected to refined filtration and chromatographic separation with continuous liquid loading at medium pressure, to obtain two eluates respectively. The eluates are concentrated to obtain beta-carotene, yellowish paprika red pigment, orange paprika red pigment, and reddish paprika red pigment respectively.

In the invention, normal phase separation is adopted in silica gel column chromatography. The order in which substances are eluted depends on the polarity of the substances. As the elution solvents are used in ascending order of polarity, and substances are also eluted off in ascending order of polarity. Therefore, beta-carotene with lowest polarity is eluted off first, sequentially followed by elution of other yellow pigment substances, reddish pigment, and orange pigment with highest polarity. By this method, the separation effect of paprika red pigments of different colors is optimized, the yellowish, orange, and reddish paprika red pigments are obtained respectively, and the absorbance ratio of the red pigment is increased from 1.02 to 1.08 or more. On the other hand, the invention also simplifies the direct separation of beta-carotene from paprika oleoresin, thereby achieving large-scale industrial production of beta-carotene.

To sum up, in the invention, by using a separation technology integrating enzymatic hydrolysis and medium-pressure column chromatography for the first time, a product containing 20% beta-carotene can be directly prepared, and the quality of the paprika red pigment product is significantly improved, thereby significantly improving the comprehensive quality and added value, and meeting the requirements for wide application in foods and health foods. The technical effects of the technical solutions of the invention not only can significantly improve the product scale and market advantage of the beta-carotene product prepared from pepper, but also lay a foundation for the further development of the pepper industry.

### DETAILED DESCRIPTION OF THE PREFERRED EMBODIMENTS

In order for those skilled in the art to better understand the invention, the invention will be described in further detail below. It should be understood that the scope of protection of the invention is not limited to the detailed description below.

The embodiments of the invention provide a method for preparing beta-carotene from pepper pigment and a product thereof, to solve the technical problem in the prior art that separation technologies for paprika red pigment cannot be industrialized due to the complex pigment composition of paprika red pigment and the difficulty of separation.

The technical solutions of the invention will be described in detail below through specific examples.

Unless otherwise particularly stated, the technical means used in the invention is conventional means well known to those skilled in the art, and raw materials, reagents, instruments, and equipment used in the invention are commercially available or can be prepared by existing methods. The reagents used in the invention are of analytical grade unless otherwise particularly stated. The paprika oleoresin used in the invention is purchased from Zhongda Hengyuan Biotechnology Stock Co., Ltd., and the preparation method is as follows: drying mature pepper at 30° C. to 50° C., followed by crushing and granulation to obtain pepper particles; conveying the pepper particles into a continuous extractor, and spraying an extraction solvent through a spraying pipe in the conveying process, so that the extraction solvent and the pepper particles are evenly mixed, and paprika red pigment is leached out through the extraction solvent, to obtain a pigment leachate; and concentrating the pigment leachate under reduced pressure so that the solvent content is less than 0.5%, to obtain the paprika oleoresin.

### Example 1

(a) 100 Kg of paprika oleoresin (with a color value of 273.34, an absorbance ratio (A_{470 nm}/A_{454 nm}) of 0.979, and a beta-carotene content of 1.07%) was weighed. Membrane filtration water was added in an amount which was 0.5 times that of the paprika oleoresin, and lipase was added in an amount which was 1.5 wt % of the paprika oleoresin. The solution was stirred and reacted at 38° C. for 10 h to obtain a paprika red pigment hydrolysate.

(b) After the enzymatic hydrolysis reaction was completed, 190 Kg of a vegetable oil extraction solvent was added into the hydrolysate to adjust the color value to 98, stirred to dissolve at room temperature for 30 min, and allowed to stand for 60 min. The aqueous phase in the lower layer was slowly removed. The pigment phase in the upper layer was filtered by an organic filter membrane of 1 μm to obtain a paprika red pigment filtrate.

(c) The paprika red pigment filtrate was separated by silica gel column chromatography with continuous liquid loading at a flow rate of 1 Bv/h and a pressure of 0.7 MPa to 0.8 MPa. Elution was carried out with 1500 L of a vegetable oil extraction solvent at a flow rate of 1 Bv/h to obtain a beta-carotene eluate. The beta-carotene eluate was concentrated under reduced pressure to obtain 4.64 Kg of an orange-yellow oleoresin, with a color value of 510.15, an absorbance ratio (A_{470 nm}/A_{454 nm}) of 0.850, and a yield of 8.68% based on paprika red pigment. The content of beta-carotene was 21.36%, and the yield was 92.63% based on the content of beta-carotene.

(d) After the elution of beta-carotene was completed, elution was carried out with 750 L of a mixed solvent of a vegetable oil extraction solvent and acetone (at a mass ratio of 10:1) at a flow rate of 1 Bv/h to obtain a yellow pigment eluate. The yellow pigment eluate was concentrated under reduced pressure to obtain 30.14 Kg of a yellow pigment oleoresin, with a color value of 107.42, an absorbance ratio (A_{470 nm}/A_{454 nm}) of 0.835, and a yield of 11.86%. The content of beta-carotene was 0.05%, and the yield was 1.52% based on the content of beta-carotene.

(e) After the elution of yellow pigments was completed, elution was carried out with 1500 L of a mixed solvent of a vegetable oil extraction solvent and acetone (at a mass ratio of 10:1) at a flow rate of 1 Bv/h to obtain a red pigment eluate. The red pigment eluate was concentrated under reduced pressure to obtain 35.97 Kg of a reddish paprika red pigment oleoresin, with a color value of 411.89, an absorbance ratio (A_{470 nm}/A_{454 nm}) of 1.080, and a yield of 54.27%.

(f) After the elution of red pigments was completed, elution was carried out with 900 L of acetone at a flow rate of 1 Bv/h to obtain an orange pigment eluate. The orange pigment eluate was concentrated under reduced pressure to obtain 18.63 Kg of an orange paprika red pigment oleoresin, with a color value of 324.44, an absorbance ratio (A_{470 nm}/A_{454 nm}) of 0.951, and a yield of 22.14%.

(g) After the elution with acetone was completed, the medium-pressure chromatographic column was treated by solvent replacement and equilibrium using a vegetable oil extraction solvent at a flow rate of 1 Bv/h. The amount of solvent used was 1500 L (2 Bv). After the treatment by solvent replacement and equilibrium was completed, separation production of a next batch could be carried out.

### Example 2

(a) 100 Kg of paprika oleoresin (with a color value of 304.17, an absorbance ratio (A_{470 nm}/A_{454 nm}) of 0.987, and a beta-carotene content of 1.09%) was weighed. Membrane filtration water was added in an amount which was 0.5 times that of the paprika oleoresin, and lipase was added in an amount which was 1.5 wt % of the paprika oleoresin. The solution was stirred and reacted at 38° C. for 10 h to obtain a paprika red pigment hydrolysate.

(b) After the enzymatic hydrolysis reaction was completed, 210 Kg of petroleum ether was added into the hydrolysate to adjust the color value to 100, stirred to dissolve at room temperature for 30 min, and allowed to stand for 60 min. The aqueous phase in the lower layer was slowly removed. The pigment phase in the upper layer was filtered by an organic filter membrane of 0.8 μm to obtain a paprika red pigment filtrate.

(c) The paprika red pigment filtrate was separated by silica gel column chromatography with continuous liquid loading at a flow rate of 1.5 Bv/h and a pressure of 1.0 MPa to 1.2 MPa. Elution was carried out with 1500 L of petroleum ether at a flow rate of 1.0 Bv/h to obtain a beta-carotene eluate. The beta-carotene eluate was concentrated under reduced pressure to obtain 4.50 Kg of an orange-yellow oleoresin, with a color value of 540.76, an absorbance ratio (A_{470 nm}/A_{454 nm}) of 0.855, and a yield of 8.01% based on paprika red pigment. The content of beta-carotene was 23.08%, and the yield was 95.34% based on the content of beta-carotene.

(d) After the elution of beta-carotene was completed, elution was carried out with 1000 L of a mixed solvent of petroleum ether and acetone (at a mass ratio of 12:1) at a flow rate of 1 Bv/h to obtain a yellow pigment eluate. The yellow pigment eluate was concentrated under reduced pressure to obtain 31.62 Kg of a yellow pigment oleoresin, with a color value of 89.22, an absorbance ratio (A_{470 nm}/A_{454 nm}) of 0.841, and a yield of 9.28% based on paprika red pigment. The content of beta-carotene was 0.10%, and the yield was 2.86% based on the content of beta-carotene.

(e) After the elution of yellow pigments was completed, elution was carried out with 2000 L of a mixed solvent of petroleum ether and acetone (at a mass ratio of 12:1) at a flow rate of 1 Bv/h to obtain a red pigment eluate. The red pigment eluate was concentrated under reduced pressure to obtain 36.48 Kg of a reddish paprika red pigment oleoresin, with a color value of 466.92, an absorbance ratio (A_{470 nm}/A_{454 nm}) of 1.083, and a yield of 56.03%.

(f) After the elution of red pigments was completed, elution was carried out with 750 L of acetone at a flow rate of 0.8 Bv/h to obtain an orange pigment eluate. The orange pigment eluate was concentrated under reduced pressure to obtain 18.31 Kg of an orange paprika red pigment oleoresin, with a color value of 361.77, an absorbance ratio (A_{470 nm}/A_{454 nm}) of 0.949, and a yield of 21.79%.

(g) After the elution with acetone was completed, the medium-pressure chromatographic column was treated by solvent replacement and equilibrium using petroleum ether at a flow rate of 1.5 Bv/h. The amount of solvent used was 2250 L (3 Bv). After the treatment by solvent replacement and equilibrium was completed, separation production of a next batch could be carried out.

### Example 3

(a) 100 Kg of paprika oleoresin (with a color value of 267.63, an absorbance ratio (A_{470 nm}/A_{454 nm}) of 0.979, and a beta-carotene content of 1.03%) was weighed. Membrane filtration water was added in an amount which was 0.5 times that of the paprika oleoresin, and lipase was added in an amount which was 1.5 wt % of the paprika oleoresin. The solution was stirred and reacted at 38° C. for 10 h to obtain a paprika red pigment hydrolysate.

(b) After the enzymatic hydrolysis reaction was completed, 180 Kg of n-hexane was added into the hydrolysate to adjust the color value to 100, stirred to dissolve at room temperature for 30 min, and allowed to stand for 60 min. The aqueous phase in the lower layer was slowly removed. The pigment phase in the upper layer was filtered by an organic filter membrane of 0.45 μm to obtain a paprika red pigment filtrate.

(c) The paprika red pigment filtrate was separated by silica gel column chromatography with continuous liquid loading at a flow rate of 2.0 Bv/h and a pressure of 1.5 MPa to 1.7 MPa. Elution was carried out with 2250 L of n-hexane at a flow rate of 1.5 Bv/h to obtain a beta-carotene eluate. The beta-carotene eluate was concentrated under reduced pressure to obtain 4.74 Kg of an orange-yellow oleoresin, with a color value of 489.23, an absorbance ratio (A_{470 nm}/A_{454 nm}) of 0.848, and a yield of 9.03% based on paprika red pigment. The content of beta-carotene was 20.38%, and the yield was 93.79% based on the content of beta-carotene.

(d) After the elution of beta-carotene was completed, elution was carried out with 600 L of a mixed solvent of n-hexane and acetone (at a mass ratio of 8:1) at a flow rate of 1 Bv/h to obtain a yellow pigment eluate. The yellow pigment eluate was concentrated under reduced pressure to obtain 30.36 Kg of a yellow pigment oleoresin, with a color value of 88.77, an absorbance ratio (A_{470 nm}/A_{454 nm}) of 0.841, and a yield of 10.07%. The content of beta-carotene was 0.03%, and the yield was 1.02% based on the content of beta-carotene.

(e) After the elution of yellow pigments was completed, elution was carried out with 1300 L of a mixed solvent of n-hexane and acetone (at a mass ratio of 8:1) at a flow rate of 1 Bv/h to obtain a red pigment eluate. The red pigment eluate was concentrated under reduced pressure to obtain 34.34 Kg of a reddish paprika red pigment oleoresin, with a color value of 416.02, an absorbance ratio (A_{470 nm}/A_{454 nm}) of 1.081, and a yield of 53.38%.

(f) After the elution of red pigments was completed, elution was carried out with 1000 L of acetone at a flow rate of 1.2 Bv/h to obtain an orange pigment eluate. The orange pigment eluate was concentrated under reduced pressure to obtain 18.49 Kg of an orange paprika red pigment oleoresin, with a color value of 338.94, an absorbance ratio (A_{470 nm}/A_{454 nm}) of 0.957, and a yield of 23.42%.

(g) After the elution with acetone was completed, the medium-pressure chromatographic column was treated by solvent replacement and equilibrium using n-hexane at a flow rate of 1.2 Bv/h. The amount of solvent used was 1875 L (2.5 Bv). After the treatment by solvent replacement and equilibrium was completed, separation production of a next batch could be carried out.

### Comparative Example 1

(a) 100 Kg of paprika oleoresin (with a color value of 267.63, an absorbance ratio/A (A_{470 nm}/A_{454 nm}) of 0.979, and a beta-carotene content of 1.03%) was weighed. 180 Kg of n-hexane was added and stirred to dissolve to adjust the color value to 96, and filtered with a 300-mesh filter cloth to obtain a paprika red pigment filtrate.

(b) The paprika red pigment filtrate was separated by normal-pressure silica gel column chromatography with continuous liquid loading at a flow rate of 1 Bv/h. The stationary phase specification of the chromatographic column was 60-100 mesh chromatographic silica gel. Elution was carried out with 1500 L of n-hexane at a flow rate of 1 Bv/h to obtain a yellow pigment eluate. The yellow pigment eluate was concentrated under reduced pressure to obtain 43.8 Kg of a dark yellow oleoresin, with a color value of 138.52, an absorbance ratio (A_{470 nm}/A_{454 nm}) of 0.887, and a yield of 22.68% based on paprika red pigment. The content of beta-carotene was 2.26%, and the yield was 96.31% based on the content of beta-carotene.

(c) After the elution of yellow pigments was completed, elution was carried out with 1500 L of acetone at a flow rate of 1 Bv/h to obtain a red pigment eluate. The red pigment eluate was concentrated under reduced pressure to obtain 54.4 Kg of a red pigment oleoresin, with a color value of 363.35, an absorbance ratio (A_{470 nm}/A_{454 nm}) of 1.021, and a yield of 73.83%.

(d) After the elution with acetone was completed, the solvent in the chromatographic column was blown out by nitrogen. The silica gel in the chromatographic column was removed, dried under reduced pressure at 60° C. and −0.06 MPa until there was no obvious solvent, and then activated at 105° C. for 2 h. Then, the column was packed, to allow for separation production of a next batch.

### Comparative Example 2

(a) 100 Kg of paprika oleoresin (with a color value of 294.38, an absorbance ratio (A_{470 nm}/A_{454 nm}) of 0.983, and a beta-carotene content of 1.05%) was weighed. Membrane filtration water was added in an amount which was 0.5 times that of the paprika oleoresin, and lipase was added in an amount which was 1.5 wt % of the paprika oleoresin. The solution was stirred and reacted at 38° C. for 10 h to obtain a paprika red pigment hydrolysate.

(b) After the enzymatic hydrolysis reaction was completed, 200 Kg of a vegetable oil extraction solvent was added into the hydrolysate to adjust the color value to 100, stirred to dissolve at room temperature for 30 min, and allowed to stand for 60 min. The aqueous phase in the lower layer was slowly removed. The pigment phase in the upper layer was filtered with a 300-mesh filter cloth to obtain a paprika red pigment filtrate.

(c) The paprika red pigment filtrate was separated by normal-pressure silica gel column chromatography with continuous liquid loading at a flow rate of 1.0 Bv/h. The stationary phase specification of the chromatographic column was 200-300 mesh chromatographic silica gel. Elution was carried out with 750 L of a vegetable oil extraction solvent at a flow rate of 1.0 Bv/h to obtain a yellow pigment eluate I. The yellow pigment eluate was concentrated under reduced pressure to obtain 11.1 Kg of an orange-yellow oleoresin, with a color value of 307.76, an absorbance ratio (A_{470 nm}/A_{454 nm}) of 0.849, and a yield of 11.63% based on paprika red pigment. The content of beta-carotene was 5.32%, and the yield was 79.48% based on the content of beta-carotene.

(d) Elution was carried out with 1500 L of a vegetable oil extraction solvent at a flow rate of 1.0 Bv/h to obtain a yellow pigment eluate II. The yellow pigment eluate II was concentrated under reduced pressure to obtain 24.51 Kg of a dark yellow oleoresin, with a color value of 147.25, an absorbance ratio (A$70 mm/A_{454 nm} of 0.891, and a yield of 12.26% based on paprika red pigment. The content of beta-carotene was 0.70%, and the yield was 16.28% based on the content of beta-carotene.

(e) After the elution of yellow pigments was completed, elution was carried out with 1500 L of acetone at a flow rate of 1 Bv/h to obtain a red pigment eluate. The red pigment eluate was concentrated under reduced pressure to obtain 53.28 Kg of a red pigment oleoresin, with a color value of 393.83, an absorbance ratio (A_{470 nm}/A_{454 nm}) of 1.024, and a yield of 71.28%.

(f) After the elution with acetone was completed, the solvent in the chromatographic column was blown out by nitrogen. The silica gel in the chromatographic column was removed, dried under reduced pressure at 60° C. and −0.06 MPa until there was no obvious solvent, and then activated at 105° C. for 2 h. Then, the column was packed, to allow for separation production of a next batch.

TABLE 1
Comparison of analytical test results in Examples 1-3 and Comparative Examples 1-2

| | | Experiment No. | | | | |
|---|---|---|---|---|---|---|
| | | | | | Comparative | Comparative |
| Test item | | Example 1 | Example 2 | Example 3 | Example 1 | Example 2 |
| Raw | Color value | 273.34 | 304.17 | 267.63 | 267.63 | 294.38 |
| material | A_{470 nm}/A_{454 nm} | 0.979 | 0.987 | 0.979 | 0.979 | 0.983 |
| | Beta-carotene | 1.07% | 1.09% | 1.03% | 1.03% | 1.05% |
| | content | | | | | |
| Yellow | Color value | 510.15 | 540.76 | 489.23 | 138.52 | 307.76 |
| pigment | A_{470 nm}/A_{454 nm} | 0.850 | 0.855 | 0.848 | 0.887 | 0.849 |
| oleoresin | Beta-carotene | 21.36% | 23.08% | 20.38% | 2.26% | 5.32% |
| I | content | | | | | |
| Yellow | Color value | 107.42 | 89.22 | 88.77 | / | 147.25 |
| pigment | A_{470 nm}/A_{454 nm} | 0.835 | 0.841 | 0.841 | / | 0.891 |
| oleoresin | Beta-carotene | 0.05% | 0.10% | 0.03% | / | 0.70% |
| II | content | | | | | |
| Red | Color value | 411.89 | 466.92 | 416.02 | 363.35 | 393.83 |
| pigment | A_{470 nm}/A_{454 nm} | 1.080 | 1.083 | 1.081 | 1.021 | 1.024 |
| oleoresin | | | | | | |
| Orange | Color value | 324.44 | 361.77 | 338.94 | / | / |
| pigment | A_{470 nm}/A_{454 nm} | 0.951 | 0.949 | 0.957 | / | / |
| oleoresin | | | | | | |

According to the analysis of the results in Table 1, it can be seen that:

(1) The paprika red pigment can be separated into three colors: yellowish (A_{470 nm}/A_{454 nm}≤0.855), orange (A_{470 nm}/A_{454 nm}=0.949 to 0.957), and reddish (A_{470 nm}/A_{454 nm}≥1.080) in the examples, but can only be separated into yellowish (A_{470 nm}/A_{454 nm}≤0.890) and reddish (A_{470 nm}/A_{454 nm}≥1.020) in the comparative examples.

(2) The A_{470 nm}/A_{454 nm} ratio of the reddish paprika red pigment can be increased to at least 1.080 in the examples, but can only reach at least 1.020 in the comparative examples, because the orange pigment cannot be separated from the red pigment.

(3) In the examples, beta-carotene with a content of 20% or more can be directly separated from paprika oleoresin having been subjected to enzymatic hydrolysis; while the beta-carotene content obtained through separation in Comparative Example 1 was only 2.31%, and the beta-carotene content obtained in Comparative Example 2 using enzymatic hydrolysis was still only 5.32%. The average beta-carotene content in the examples was more than 9 times of that in Comparative Example 1, and more than 4 times of that in Comparative Example 2.

Based on the above analysis results, it can be seen that the advantages of the processes of the examples in separation effect, product quality and the like are obvious, and the product containing 20% beta-carotene produced by the invention can be widely applied to foods and health foods.

The foregoing descriptions of specific examples of the invention are for purposes of explanation and illustration. These descriptions are not intended to limit the invention to the precise form disclosed and it is apparent that various changes and variations may be made in accordance with the above teachings. The examples are selected and described for the purpose of explaining particular principles and practical applications of the invention, to enable those skilled in the art to practice the examples and make different selections and variations to the examples. The scope of the present invention is defined by the appended claims and their equivalents.

1. A method for preparing a beta-carotene from pepper pigment, comprising:
(1) enzymatic hydrolysis of paprika oleoresin to obtain a paprika red pigment hydrolysate;
(2) extraction: dissolving the paprika red pigment hydrolysate obtained in the step (1) in an alkane solvent, standing for layering, and collecting the supernatant to obtain a paprika red pigment solution;
(3) refined filtration: filtering the paprika red pigment solution obtained in the step (2) by a filter membrane to obtain a paprika red pigment filtrate;
(4) adsorption: carrying out chromatographic separation of the paprika red pigment filtrate obtained in the step (3) with a medium-pressure silica gel chromatographic column, and collecting an effluent from the chromatographic column;
(5) elution of beta-carotene: after loading onto the medium-pressure chromatographic column is completed, eluting with a first eluent, and collecting and concentrating the eluate to obtain beta-carotene oleoresin;
(6) elution of yellow pigments: after the elution of beta-carotene is completed, eluting with a second eluent, and concentrating the eluate to obtain a yellow pigment oleoresin;
(7) elution of red pigments: after the elution of yellow pigments is completed, eluting with a third eluent, and concentrating the eluate to obtain reddish paprika red pigment; and
(8) elution of orange pigments: after the elution of red pigments is completed, eluting the silica gel column with a fourth eluent, concentrating the eluate, and mixing the concentrated eluate with the yellow pigment oleoresin to obtain orange paprika red pigment.

2. The method for preparing the beta-carotene from pepper pigment, according to claim 1, wherein after the step (8) is completed, the method further comprises a step (9) of solvent replacement and equilibrium:
treating the medium-pressure silica gel chromatographic column by solvent replacement and equilibrium using a replacement solvent, to allow for separation production of a next batch.

3. The method for preparing the beta-carotene from pepper pigment, according to claim 1, wherein in the step 1, the enzymatic hydrolysis specifically comprises: adding membrane filtration water and lipase into the paprika oleoresin, stirring and reacting the mixture.

4. The method for preparing the beta-carotene from pepper pigment, according to claim 1, wherein in the step (2), the alkane solvent is an alkane containing 4-7 carbon atoms; and the alkane solvent is added in an amount to adjust the color value of the paprika red pigment solution to 60-120.

5. The method for preparing the beta-carotene from pepper pigment, according to claim 1, wherein in the step (4), the stationary phase of the medium-pressure silica gel chromatographic column is 300-500 mesh chromatographic silica gel, and the mass ratio of the amount of silica gel to the paprika oleoresin is 1:(0.25-0.4).

6. The method for preparing the beta-carotene from pepper pigment, according to claim 1, wherein the first eluent is an alkane containing 4-7 carbon atoms, the elution volume is 1-5 BV, and the elution flow rate is 0.5-3 BV/h.

7. The method for preparing the beta-carotene from pepper pigment, according to claim 1, wherein in the step (6), the second eluent is obtained by mixing an alkane containing 4-7 carbon atoms and acetone according to a mass ratio of (8-50):1; and
in the step (7), the third eluent is obtained by mixing an alkane containing 4-7 carbon atoms and acetone according to a mass ratio of (8-50):1.

8. The method for preparing the beta-carotene from pepper pigment, according to claim 1, wherein in the step (8), the fourth eluent is acetone, the elution volume is 1-5 BV, and the elution flow rate is 0.5-3 BV/h.

9. The method for preparing the beta-carotene from pepper pigment, according to claim 2, wherein in the step (9), the replacement solvent is an alkane containing 4-7 carbon atoms, the solvent replacement and equilibrium volume is 1.5-5 BV, and the flow rate is 0.5-3 BV/h.

10. The method for the preparing beta-carotene from pepper pigment according to claim 1, wherein the yield of the beta-carotene is 92.5% or above, with a purity of 20% or above.
